(19) United States
(12) Patent Application Publication
(10) Pub. No.: US 2024/0344849 A1
(43) Pub. Date: Oct. 17, 2024

(54) INFORMATION PROCESSING APPARATUS, INFORMATION PROCESSING METHOD, PROGRAM, AND INFORMATION PROCESSING SYSTEM

(71) Applicant: SONY GROUP CORPORATION, TOKYO (JP)

(72) Inventor: YU SHIGETA, TOKYO (JP)

(21) Appl. No.: 18/294,257

(22) PCT Filed: Feb. 28, 2022
(86) PCT No.: PCT/JP2022/008260

§ 371 Date: Feb. 1, 2024

(30) Foreign Application Priority Data
Aug. 16, 2021 (JP) 2021-132354

(51) Int. Cl. G01C 22/00; G16H 20/30

(52) U.S. Cl. CPC G01C 22/00; G16H 20/30

(57) ABSTRACT

The present technology relates to an information processing apparatus, an information processing method, a program, and an information processing system that enables walking information of an elderly person to be more accurately acquired. The information processing apparatus calculates walking information indicating a walking state of a user by using position information of a consecutive section set on the basis of behavior analysis information of the user. The present technology can be applied to a walking information providing system of the user.

### TECHNICAL FIELD

The present technology relates to an information processing apparatus, an information processing method, a program, and an information processing system, in particular to an information processing apparatus, an information processing method, a program, and an information processing system that enable user walking information to be more accurately acquired.

### BACKGROUND ART

At present, the birthrate is declining and the population is aging, and the frailty (weakness) of the elderly is becoming a serious concern.

In order to detect a sign of frailty at an early stage, it is very important to detect a change in a walking state by constantly measuring an exercise amount, a walking distance, and the like of an elderly person.

At the time of calculating the number of steps in a pedometer, a step length cannot be dynamically calculated, and conventionally, a method of multiplying the height by a constant such as height×0.45 has been adopted. However, the method is difficult to be applied to an elderly person with a small step length, who has difficulty in walking normally.

Patent Document 1 describes a technique in which a specific area is detected using a global positioning system (GPS) or the like, and the step length is measured in a case where the person is in the specific area.

However, in a terminal using the GPS only, a moving distance indoors is difficult to be calculated.

### CITATION LIST

### Patent Document

Patent Document 1: Japanese Patent Application Laid-Open No. 2016-211954

### SUMMARY OF THE INVENTION

### Problems to be Solved by the Invention

As described above, a method of accurately acquiring the walking information of the user is urgently required in order to detect a sign of frailty.

The present technology has been made in view of such a situation, and an object of the present technology is to enable walking information of an elderly person to be more accurately acquired.

### Solutions to Problems

An information processing apparatus according to a first aspect of the present technology includes a walking information calculation unit that calculates walking information indicating a walking state of a user by using position information of a consecutive section set on the basis of behavior analysis information of the user.

In the first aspect of the present technology, the walking information indicating the walking state of the user is calculated by using the position information of the consecutive section set on the basis of the behavior analysis information of the user.

An information processing apparatus according to a second aspect of the present technology includes: a behavior analysis unit that analyzes behavior of a user; and a transmission unit that, in a case where specific behavior has been analyzed, sets position information of a consecutive section in which the specific behavior has been analyzed, and transmits the position information to another information processing apparatus.

In the second aspect of the present technology, the behavior analysis of the user is performed, and in a case where the specific behavior has been analyzed, the position information of the consecutive section in which the specific behavior has been analyzed is set, and the position information is transmitted to another information processing apparatus.

An information processing system according to a third aspect of the present technology includes: a first information processing apparatus including a behavior analysis unit that analyzes behavior of a user, and a transmission unit that sets, in a case where specific behavior has been analyzed, position information of a consecutive section in which the specific behavior has been analyzed and transmits the position information to a second information processing apparatus; and the second information processing apparatus including a walking information calculation unit that calculates walking information indicating a walking state of the user by using the position information of the consecutive section set by the first information processing apparatus.

In the third aspect of the present technology, the behavior analysis of the user is performed by the first information processing apparatus, and in a case where the specific behavior has been analyzed, the position information of the consecutive section in which the specific behavior has been analyzed is set, and the position information is transmitted to the second information processing apparatus. The second information processing apparatus calculates the walking information indicating the walking state of the user by using the position information of the consecutive section set by the first information processing apparatus.

### BRIEF DESCRIPTION OF DRAWINGS

FIG. 1 is a diagram illustrating a configuration of an embodiment of a walking information providing system to which the present technology is applied.

FIG. 2 is a block diagram illustrating a hardware configuration example of a device.

FIG. 3 is a block diagram illustrating a main functional configuration example of the device, a server, and a terminal.

FIG. 4 is a flowchart for explaining behavior analysis processing in the device.

FIG. 5 is a flowchart for explaining walking information calculation processing in the server.

FIG. 6 is a flowchart for explaining distance calculation processing in a position information consecutive section in step S33 in FIG. 5.

FIG. 7 is a block diagram illustrating a second configuration example of the walking information calculation unit.

FIG. 8 is a flowchart for explaining another example of the walking information calculation processing in the server.

FIG. 9 is a diagram for explaining an extraction method of an area section.

FIG. 10 is a block diagram illustrating a third configuration example of the walking information calculation unit.

FIG. 11 is a flowchart for explaining another example of the walking information calculation processing in the server.

FIG. 12 is a flowchart describing estimation processing of a height of stairs executed in step S74 in FIG. 11.

FIG. 13 is a diagram illustrating a calculation method of the height of stairs.

FIG. 14 is a block diagram illustrating another functional configuration example of the device and the server.

FIG. 15 is a diagram illustrating a configuration of an embodiment of a health information providing system to which the present technology is applied.

FIG. 16 is a flowchart for explaining processing by the device and the server.

FIG. 17 is a diagram illustrating a configuration example of a health information providing system.

### MODE FOR CARRYING OUT THE INVENTION

Hereinafter, a mode for carrying out the present technology is described. The description is given in the following order.
1. First embodiment (Walking information providing system)
2. Second embodiment (Health information providing system)
3. Others

### 1. First Embodiment (Walking Information Providing System)

### (Configuration Example of Walking Information Providing System)

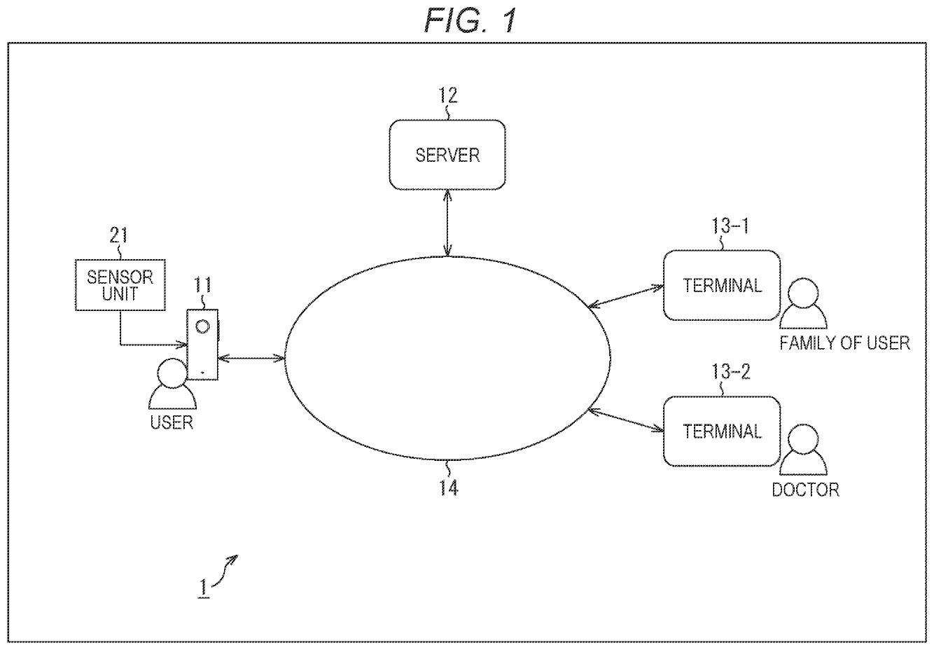

FIG. 1 is a diagram illustrating a configuration of an embodiment of a walking information providing system to which the present technology is applied.

At present, the birthrate is declining and the population is aging, and the frailty (weakness) of the elderly is becoming a serious concern.

In order to detect a sign of frailty at an early stage, it is very important to detect a change in a walking state by constantly measuring an exercise amount, a walking distance, and the like of an elderly person and obtaining walking information such as the exercise amount and the walking distance indicating the walking state.

A walking information providing system 1 in FIG. 1 is a system that acquires the walking information of a user by using sensor data that is information obtained by sensing an elderly user, and notifies, for example, the user, a family member of the user, a doctor, and the like of the walking information of the user. Note that the user may not be an elderly person.

The walking information providing system 1 includes a device 11, a server 12, and terminals 13-1 and 13-2. The device 11, the server 12, and the terminals 13-1 and 13-2 are connected to each other via the Internet 14.

Note that the terminals 13-1 and 13-2 are collectively referred to as terminals 13 in a case where the terminals do not need to be distinguished from each other.

The device 11 is a small wireless communication device having a wireless communication function, and includes a sensor unit 21.

The sensor unit 21 includes an acceleration sensor, a gyro sensor, an electronic compass, an atmospheric pressure sensor, a humidity sensor, a temperature sensor, a position information sensor such as a global navigation satellite system (GNSS), a heart rate sensor, a microphone, and the like. Note that these sensors are examples, and the device 11 may include sensors other than these sensors, or may not include any of these sensors.

The device 11 is, for example, a device that is assumed to be possessed by a user such as an elderly person and to observe behavior of the user possessing the device. Therefore, the device 11 is formed as a small and lightweight device so as not to be burdensome even if being carried by such a user. The device 11 may include a smartphone, a mobile terminal, or the like including the sensor unit 21 and a sensor corresponding thereto.

The device 11 analyzes the behavior of the user using the sensor data acquired by the sensor unit 21, and obtains behavior pattern information indicating the identified behavior pattern as a result of the behavior analysis. The device 11 transmits the behavior pattern information and the sensor data including the position information and the number of steps information to the server 12.

Furthermore, the device 11 receives and displays walking information of the user transmitted from the server 12, an amount of change in walking information which is a comparison result of pieces of daily walking information, and the like. The walking information includes, for example, information indicating a step length, the number of steps, a walking speed, a walking distance, an exercise amount, and the like. With this arrangement, the user can know one's walking state and one's behavior change.

The server 12 includes a computer or the like. The server 12 calculates the walking information by using the behavior pattern information and the sensor data transmitted from the device 11, compares the pieces of daily walking information, and calculates the amount of change in the walking information.

The server 12 transmits the calculated walking information, the amount of change in the walking information, or the like to the device 11 and the terminal 13.

The terminal 13 includes a mobile terminal, a smartphone, a tablet terminal, a personal computer, or the like.

For example, the terminal 13-1 is carried by a family member of the user. The terminal 13-1 receives and displays the walking information of the user, the amount of change in the walking information, and the like transmitted from the server 12. With this arrangement, even if the family member of the user lives far away from the user, the family member can know the walking state of the user and the behavior change of the user.

For example, the terminal 13-2 is carried by a doctor or a health consultant. The terminal 13-2 displays the walking information of the user, the amount of change in the walking information, and the like transmitted from the server 12. With this arrangement, the doctor and the health consultant can know the walking state of the user and the behavior change of the user, and can give advice and the like to the user.

As described above, according to the walking information providing system 1, because the sign of frailty of the user can be noticed at an early stage, it is possible to take measures against frailty and prevent frailty.

### <Configuration of Device>

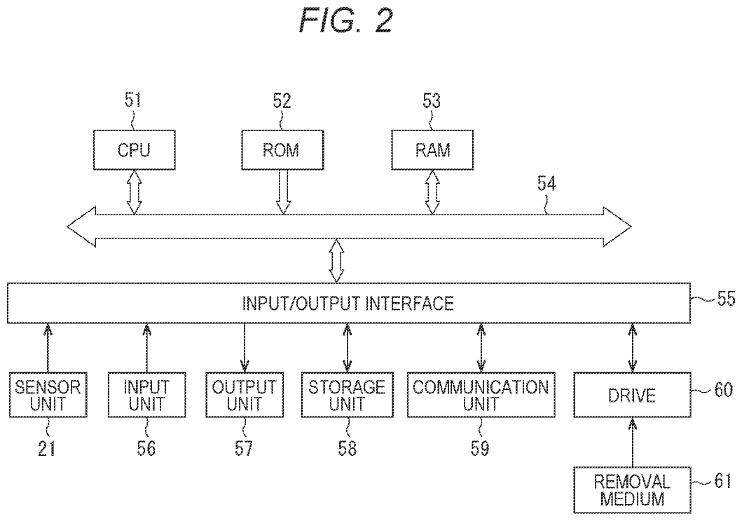

FIG. 2 is a block diagram illustrating a hardware configuration example of the device 11.

A central processing unit (CPU) 51, a read only memory (ROM) 52, and a random access memory (RAM) 53 are mutually connected by a bus 54. Moreover, an input/output interface 55 is connected to the bus 54.

The sensor unit 21, an input unit 56 including a keyboard, a mouse, and the like, and an output unit 57 including a display, a speaker, and the like are connected to the input/output interface 55. Furthermore, a storage unit 58 including a hard disk, a nonvolatile memory, and the like, a communication unit 59 including a network interface and the like to each device connected via the Internet 14, and a drive 60 that drives a removable medium 61 are connected to the input/output interface 55.

In the computer configured as described above, for example, the CPU 51 loads a program stored in the storage unit 58 into the RAM 53 via the input/output interface 55 and the bus 54 and executes the program, to perform a series of processing. Furthermore, transmission and reception of data with each device are performed via a communication unit 59.

Note that a configuration in which the sensor unit 21 is removed from the configuration illustrated in FIG. 2 is also included in the server 12 and the terminal 13.

### <Functional Configuration of Each Device>

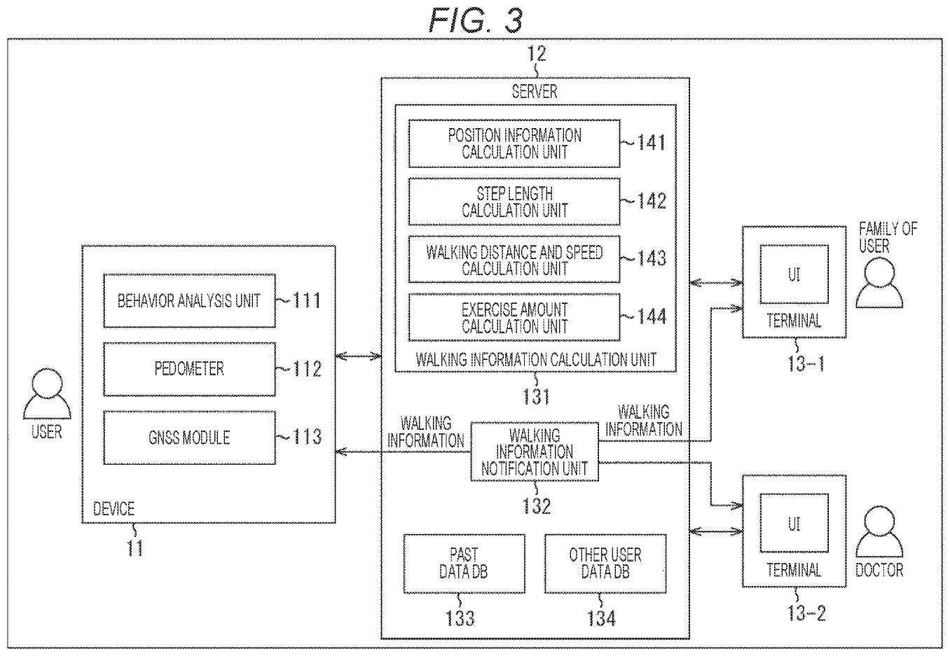

FIG. 3 is a block diagram illustrating a functional configuration example of the device 11, the server 12, and the terminals 13.

The functional configuration illustrated in FIG. 3 is realized by executing a predetermined program by the CPU 51 illustrated in FIG. 2 included in each of the devices.

The device 11 has a configuration including a behavior analysis unit 111, a pedometer 112, and a GNSS module 113.

In a case where the acceleration is detected on the basis of the sensor data supplied from the sensor unit 21, the behavior analysis unit 111 performs behavior analysis and walking analysis, and transmits sensor data including information corresponding to the behavior analysis result and the walking distance to the server 12.

By the behavior analysis, the behavior of the user is identified as a first behavior pattern, for example, three patterns of Run, Walk, and Stay. Moreover, by the detailed behavior analysis, the behavior of the user is identified as a second behavior pattern, for example, any one of the behavior patterns of Run, Walk, Stay, Stop, Bicycle, Motorcycle, Car, Train, Stairs, Escalator, and Elevator. Note that Stairs, Escalator, and Elevator are further identified as Up and Down. For example, in a case where the user is walking in a train, the behavior of the user is identified as Walk as the first behavior pattern, but is identified as Train as the second action pattern. The behavior analysis may be identification of any behavior pattern, but from the viewpoint of power saving, it is desirable that the behavior analysis unit 111 first perform the first behavior pattern and identify the second behavior pattern as necessary.

In a case where the behavior pattern is analyzed as Run or Walk and the movement distance is 7 m or more, the behavior analysis unit 111 temporarily accumulates the behavior pattern information and the sensor data including the number of steps information and the position information in the memory, accumulates the information for several hours, and then transmits the information to the server 12. Note that the time of accumulation can be set, and may be, for example, two hours or one day. Furthermore, the threshold for determining the moving distance is not necessarily limited to 7 m, and this numerical value can be variably set as appropriate.

In a case where the behavior pattern is analyzed as Run or Walk and the movement distance is less than 7 m, or in a case where the behavior pattern is other than Run and Walk, the behavior analysis unit 111 temporarily accumulates the behavior pattern information indicating the behavior pattern and the sensor data including the number of steps information in the memory, accumulates the information for several hours, and then transmits the information to the server 12.

That is, the sensor data supplied from the sensor unit 21 is also transmitted as necessary.

The pedometer 112 counts the number of steps on the basis of the sensor data supplied from the sensor unit 21, and supplies the number of steps information indicating the counted number of steps to the behavior analysis unit 111.

The GNSS module 113 is a module for acquiring current position information of the device 11 from the position information sensor of the sensor unit 21. In response to an instruction from the behavior analysis unit 111, the GNSS module 113 acquires the current position information of the device 11 from the position information sensor, and supplies the acquired position information to the behavior analysis unit 111.

The server 12 includes a walking information calculation unit 131, a walking information notification unit 132, a past data database (DB) 133, and other user data DB 134.

The walking information calculation unit 131 calculates the walking information by using the information and the sensor data transmitted from the device 11.

The walking information calculation unit 131 includes a position information calculation unit 141, a step length calculation unit 142, a walking distance and speed calculation unit 143, and an exercise amount calculation unit 144.

The position information calculation unit 141 acquires the position information at the same time as when the behavior pattern is Walk or Run from the information transmitted from the device 11 and the sensor data. By using the acquired position information, the position information calculation unit 141 sets a position information consecutive section, which is a section in which pieces of the position information appears consecutively, and calculates a distance of the position information consecutive section.

The step length calculation unit 142 acquires the number of steps information in the position information consecutive section set by the position information calculation unit 141 from the information and the sensor data transmitted from the device 11, and calculates the step length on the basis of the number of steps information in the position information consecutive section.

The walking distance and speed calculation unit 143 calculates the walking distance and the walking speed in the position information consecutive section on the basis of the step length calculated by the step length calculation unit 142.

The exercise amount calculation unit 144 calculates the exercise amount of the user from the calculated walking distance and walking speed in the position information consecutive section.

The walking information notification unit 132 compares the calculated walking information with the past walking information of the user stored in the past data DB 133 or the walking information of other users stored in the other user data DB 134. The walking information notification unit 132 generates, for example, user interface (UI) data including walking information in a case where it is determined that the amount of change is larger than a predetermined threshold value as a result of comparison with the past walking information. The walking information notification unit 132 transmits the generated UI data to the device 11 and the terminals 13, and notifies the user, a family member of the user, and the like of the walking information.

Note that, as the predetermined threshold value for determining the magnitude of the amount of change, different values may be used depending on the person who possesses the terminal 13 notified of the walking information. With this arrangement, the family of the user can be notified of the walking information in a case where there is a large change in the walking information, and the doctor can be notified even in a case where there is a slight change.

The past data DB 133 stores the past walking information for each user.

In the other user data DB 134, the past walking information of other users (for example, a general elderly person and a user diagnosed as frailty) is stored.

Note that the position information calculation unit 141 cannot obtain the position information from the GNSS module 113 of the device 11 when the user is indoors or the like.

In this case, the position information calculation unit 141 interpolates the walking information in the section in which the position information cannot be obtained (hereinafter, referred to as a GNSS unacquirable section) with reference to the walking information calculated in the section in which the position information can be obtained, the past walking information of the user in the past data DB 133, and the past walking information of other users in the other user data DB 134.

With this arrangement, the server 12 can acquire the walking information even in a section where the position information cannot be obtained.

The terminal 13 receives the UI data transmitted from the server 12 and including the walking information in a case where it is determined that the amount of change is large, and displays the UI of the received UI data.

With this arrangement, in a case where there is a variation in the daily walking state of the user, the family member of the user or the doctor who holds the terminal 13 can immediately grasp the variation in the walking state.

Note that, for example, the terminal 13-2 browsed by the doctor may be notified of the UI data including all the calculated walking information. In this case, because the daily walking information can be confirmed, the doctor can give health advice and the like to the user.

Note that, although FIG. 3 illustrates an example in which the behavior analysis unit 111 is included in the device 11, the behavior analysis unit 111 may be included in the server 12. That is, the behavior analysis may be performed by the device 11 or the server 12.

For example, in a case where the behavior analysis is performed by the server 12, the sensor data including the number of steps information and the position information is transmitted to the server 12 as it is.

Furthermore, although FIG. 3 illustrates an example in which the walking information calculation unit 131 is included in the server 12, the walking information calculation unit 131 may be included in the device 11. That is, the calculation processing of the walking information may be performed by the server 12 or the device 11.

At this time, for example, among the walking information, the step length and the exercise amount may be calculated by the server 12, and the walking distance and the walking speed may be calculated by the device 11. That is, a part of the walking information may be calculated by the server 12, and the rest may be calculated by the device 11.

### <Processing in Device>

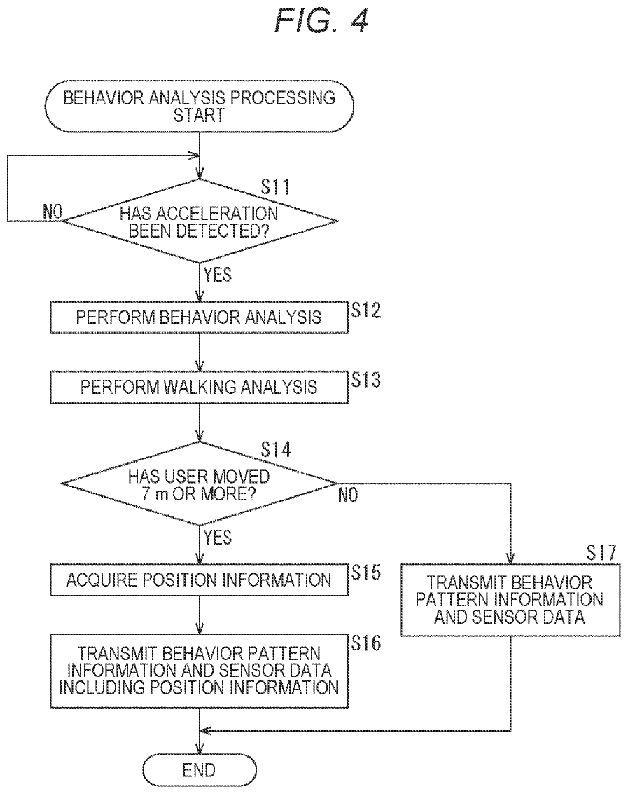

FIG. 4 is a flowchart for explaining behavior analysis processing in the device 11.

In step S11, the behavior analysis unit 111 monitors the sensor data supplied from the sensor unit 21 and waits until it is determined that acceleration has been detected. In a case where it is determined in step S11 that the acceleration has been detected, the process proceeds to step S12.

In step S12, the behavior analysis unit 111 performs the behavior analysis of the user.

In step S13, the behavior analysis unit 111 performs the walking analysis of the user, such as whether or not the user is moving and how much the user is moving, on the basis of the result of the behavior analysis.

In step S14, the behavior analysis unit 111 determines whether or not the user has moved 7 m or more on the basis of the result of the walking analysis in step S13. In a case where it is determined that the user has moved 7 m or more, the process proceeds to step S15.

In step S15, the behavior analysis unit 111 causes the GNSS module 113 to acquire position information.

In step S16, the behavior analysis unit 111 transmits the behavior pattern information and the sensor data including the position information acquired in step S15 to the server 12.

That is, the behavior analysis unit 111 temporarily accumulates the behavior pattern information and the sensor data including the number of steps information and the position information in the memory, accumulates the behavior pattern information and the sensor data for several hours, and then transmits the same to the server 12.

In a case where it is determined in step S14 that the user has not moved 7 m or more, that is, a distance that the user has moved is less than 7 m, the process proceeds to step S17.

In step S16, the behavior analysis unit 111 temporarily accumulates the behavior pattern information indicating the behavior pattern and the sensor data including the number of steps information in the memory, accumulates the behavior pattern information and the sensor data for several hours, and then transmits the same to the server 12.

After step S16 or S17, the behavior analysis processing ends.

### <Processing in Server>

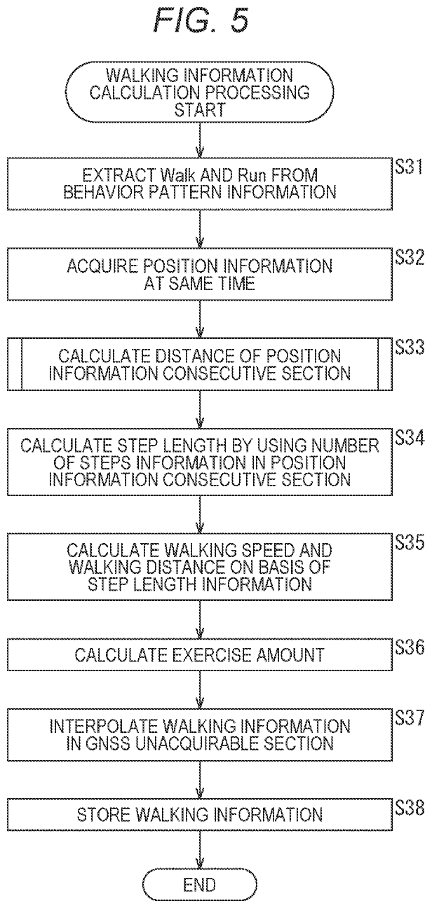

FIG. 5 is a flowchart for explaining walking information calculation processing in the server 12.

In step S31, the position information calculation unit 141 extracts the behavior pattern information of Walk or Run from the behavior pattern information transmitted from the device 11.

In step S32, the position information calculation unit 141 acquires the position information at the same time as when the behavior pattern information is Walk or Run from the sensor data transmitted from the device 11.

Note that, in a case where there is no positional information at the same time, the positional information at a time close to the time is acquired. For example, the presence of the position information is searched at an interval of one minute from the time, and in a case where there is no position information, the presence of the position information at the next interval of one minute is searched.

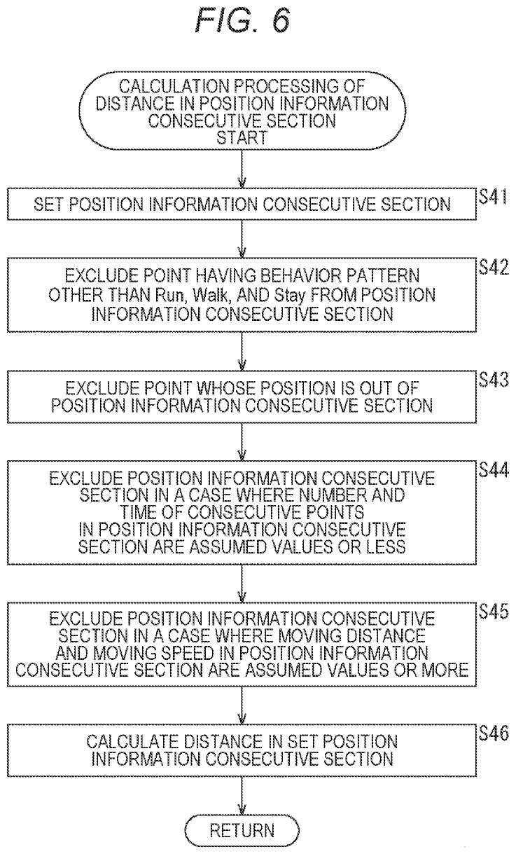

In step S33, by using the acquired position information, the position information calculation unit 141 sets a position information consecutive section, and calculates a distance of the position information consecutive section. Note that the details of processing in step S33 will be described later with reference to FIG. 6.

In step S34, the step length calculation unit 142 acquires the number of steps information in the position information consecutive section calculated by the position information calculation unit 141 from the information transmitted from the device 11, and calculates the step length on the basis of the number of steps information in the position information consecutive section.

In step S35, the walking distance and speed calculation unit 143 calculates the walking distance and the walking speed in the position information consecutive section on the basis of the step length calculated by the step length calculation unit 142.

In step S36, the exercise amount calculation unit 144 calculates the exercise amount of the user from the calculated walking distance and walking speed in the position information consecutive section.

In step S37, the position information calculation unit 141 interpolates the walking information in the GNSS unacquirable section on the basis of the walking information calculated in the section where the position information can be obtained. For example, the walking distance and the walking speed in the GNSS unacquirable section are calculated from the calculated number of steps and the step length.

In step S38, the past data DB 133 and the other user data DB 134 store the calculated walking information. The walking information is stored in association with, for example, at least one of the position information or the behavior pattern information in the position information consecutive section.

As described above, in the walking information providing system 1, the walking information regarding the walking of the user is calculated by using the position information of the consecutive section identified on the basis of the behavior pattern information of the analysis result of the behavior of the user.

With this arrangement, the walking information of the user can be acquired more accurately.

### <Calculation Processing of Distance of Position Information Consecutive Section>

FIG. 6 is a flowchart for explaining distance calculation processing in the position information consecutive section in step S33 in FIG. 5.

In step S41, by using the acquired position information, the position information calculation unit 141 sets a position information consecutive section, which is a section where the positions are consecutive.

In step S42, in a case where there is a point having the behavior pattern other than Run, Walk, and Stay as the second behavior pattern in the position information consecutive section, the position information calculation unit 141 excludes the point from the position information consecutive section.

In step S43, in a case where there is a point that is out of the position in the consecutive section indicated by the position information consecutive section, the position information calculation unit 141 excludes the point from the position information consecutive section.

Here, a point that is “out of position” indicates a point at which, by referring to a signal intensity ratio with respect to noise of the GNSS, a positional accuracy variation is a predetermined magnitude or more.

The position information calculation unit 141 sets in advance an assumed value of the number of consecutive points (for example, six points) and an assumed value of time (for example, 10 minutes). In step S44, in a case where the number of consecutive points and the time in the position information consecutive section are equal to or less than the respective assumed values, the position information calculation unit 141 excludes the position information consecutive section.

The position information calculation unit 141 sets in advance an assumed value of the moving distance or an assumed value of the moving speed for each of Run and Walk in units of pieces of behavior analysis information. In step S45, in a case where the moving distance or the moving speed in the position information consecutive section is equal to or more than the assumed value, the position information calculation unit 141 excludes the position information consecutive section.

In step S46, the position information calculation unit 141 calculates the distance of the set position information consecutive section.

In the above description, for example, the processing in steps S42 to S45 is excluding processing of position information in which inaccurate position information among the position information of the GNSS is excluded. With this arrangement, because the inaccurate position information can be excluded from the position information of the GNSS, the walking information calculation processing can be more accurately performed.

Note that the excluding processing in steps S42 to S45 in FIG. 6 are not necessarily all performed. Furthermore, the processing in step S42 is processing in a case where the behavior pattern acquired in step S31 in FIG. 5 is the first behavior pattern, and is processing unnecessary in a case where the behavior pattern acquired in step S31 is the second behavior pattern.

### <Second Configuration of Walking Information Calculation Unit>

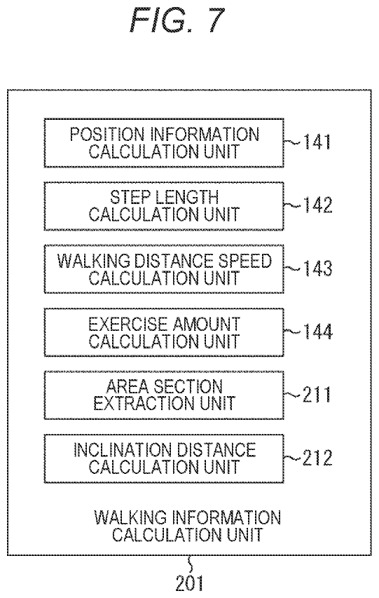

FIG. 7 is a block diagram illustrating a second configuration example of the walking information calculation unit.

A walking information calculation unit 201 in FIG. 7 is different from the walking information calculation unit 131 in FIG. 3 in that an area section extraction unit 211 and an inclination distance calculation unit 212 are added. In FIG. 7, the same portions as those in FIG. 3 are denoted by the same reference signs, and descriptions thereof are omitted.

That is, the area section extraction unit 211 refers to external environment information such as weather, temperature, time, road width, and slope (inclination) acquired via the Internet 14, and extracts, for example, a section that is a slope as an area section.

In a case where there is an area section extracted by the area section extraction unit 211 in the position information consecutive section set by the position information calculation unit 141, the inclination distance calculation unit 212 calculates the inclination distance on the basis of the external environment information of the area section.

The step length calculation unit 142 calculates the step length on the basis of the inclination distance calculated by the inclination distance calculation unit 212 and the number of steps information in the position information consecutive section.

### <Processing in Server>

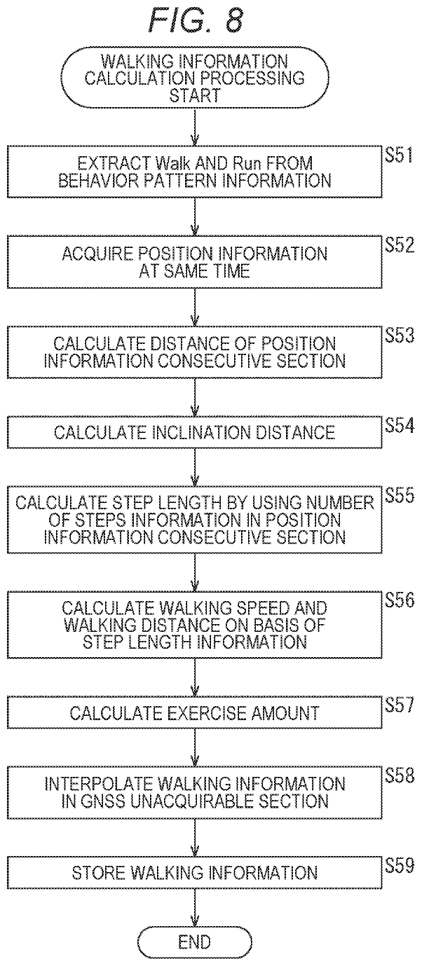

FIG. 8 is a flowchart for explaining another example of the walking information calculation processing in the server 12.

Because processing in steps S51 to S53 and steps S56 to S59 in FIG. 8 is similar to the processing in steps S31 to S33 and steps S35 to S38 in FIG. 5, descriptions thereof are omitted.

In step S54, the area section extraction unit 211 extracts the area section from the position information consecutive section set by the position information calculation unit 141 on the basis of the external environment information. The inclination distance calculation unit 212 calculates the inclination distance on the basis of the external environment information in the area section extracted by the area section extraction unit 211.

In step S55, the step length calculation unit 142 calculates the step length on the basis of the inclination distance calculated by the inclination distance calculation unit 212 and the number of steps information in the position information consecutive section.

As described above, because the inclination distance can be calculated by using the external environment information from the outside, the step length can be calculated more accurately.

Note that, in the examples of FIGS. 7 and 8, the area section is extracted on the basis of the external environment information. However, in a case where another user having the same device 11 has traveled the same route in the past, the area section may be extracted from the past walking information of other users in the other user data DB 134.

### <Extraction Method of Area Section>

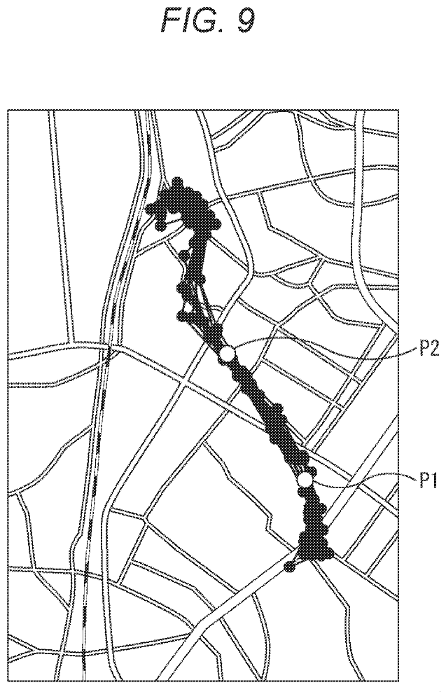

FIG. 9 is a diagram for explaining an extraction method of the area section.

In FIG. 9, black dots on the map indicate positions where other users had moved in the past and position information had been acquired. Lines connecting the black dots represent routes traveled by other users in the past. Such past route information and position information of other users are stored in the other user data DB 134 together with the walking information.

Now, when the user carrying the device 11 moves on any route including a point P1 and a point P2 shown on the map, for example, the position information calculation unit 141 acquires the position information of two points, the point P1 and the point P2. At this time, the area section extraction unit 211 extracts a section between the points P1 and P2 as an area section on the basis of the position information of the points P1 and P2 and the past walking information of other users in the other user data DB 134. Then, the position information calculation unit 141 interpolates the walking information of the area section from the past walking information of other users in the other user data DB 134.

Note that, in FIG. 9, an example of interpolating the walking information in the area section from the past walking information of other users in the other user data DB 134 has been described. However, similarly, the walking information in the area section may be interpolated from the past walking information of the user in the past data DB 133.

As described above, even in a case where an amount of the acquired position information is small, a decrease in calculation accuracy of the walking information can be suppressed.

### <Third Configuration of Walking Information Calculation Unit>

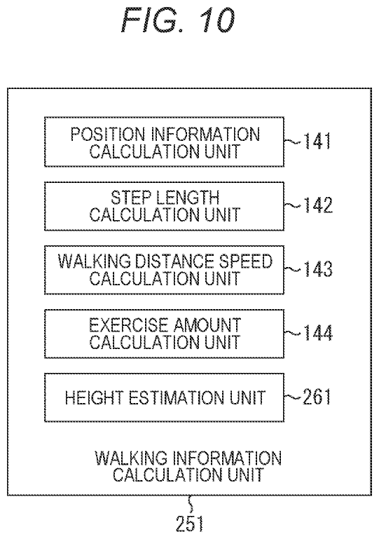

FIG. 10 is a block diagram illustrating a third configuration example of the walking information calculation unit.

The walking information calculation unit 251 in FIG. 10 is different from the walking information calculation unit 131 in FIG. 3 in that a height estimation unit 262 is added. In FIG. 10, the same portions as those in FIG. 3 are denoted by the same reference signs, and descriptions thereof are omitted.

That is, the height estimation unit 262 estimates the height on the basis of the amount of change in the atmospheric pressure by using, for example, sensor data of an atmospheric pressure sensor among the sensor data transmitted from the device 11, and outputs height information indicating the estimated height to the step length calculation unit 142.

The step length calculation unit 142 calculates the step length on the basis of the height information supplied from the height estimation unit 262 and the number of steps information in the position information consecutive section.

### <Processing in Server>

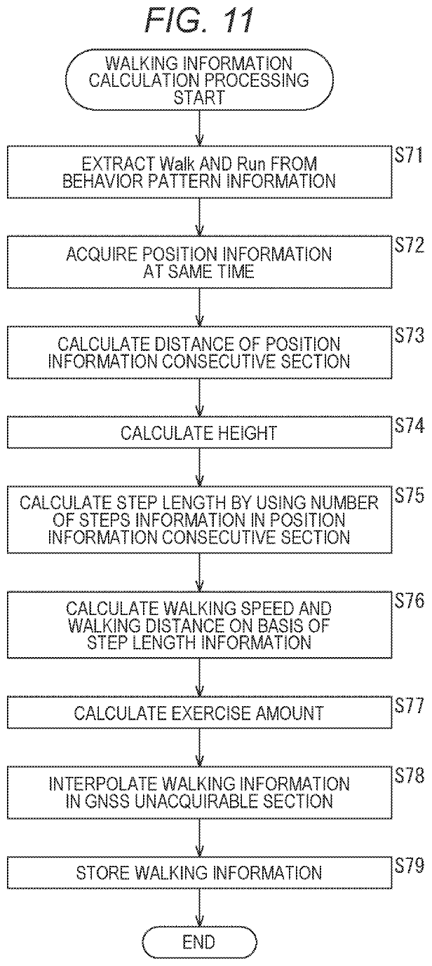

FIG. 11 is a flowchart for explaining another example of the walking information calculation processing in the server 12.

Because processing in steps S71 to S73 and steps S76 to S79 in FIG. 11 is similar to the processing in steps S31 to S33 and steps S35 to S38 in FIG. 5, descriptions thereof are omitted.

In step S74, the height estimation unit 262 estimates the height by using the sensor data of the atmospheric pressure sensor among the sensor data transmitted from the device 11, and outputs the height information indicating the estimated height to the step length calculation unit 142.

In step S75, the step length calculation unit 142 calculates the step length on the basis of the height information supplied from the height estimation unit 262 and the number of steps information in the position information consecutive section.

As described above, because the height of a slope, stairs, or the like during traveling can be estimated by using the sensor data acquired from the atmospheric pressure sensor or the like of the device 11, the step length can be calculated more accurately.

### <Estimation Processing of Height of Stairs>

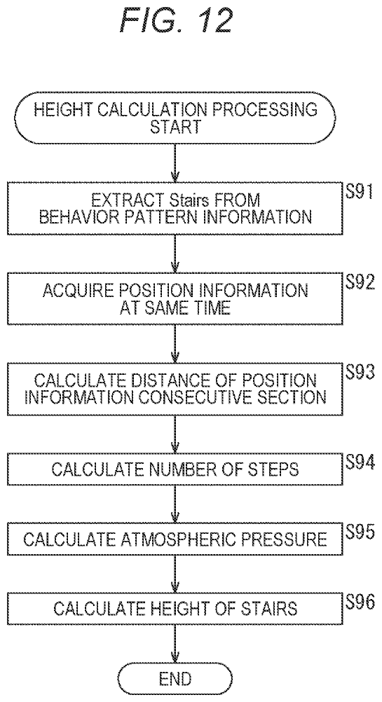

FIG. 12 is a flowchart describing, for example, estimation processing of a height of stairs executed in step S74 in FIG. 11.

In step S91, the position information calculation unit 141 extracts the behavior pattern information of Stairs from the behavior pattern information transmitted from the device 11.

In step S92, the position information calculation unit 141 acquires the position information at the same time as when the behavior pattern information is Stairs from the sensor data transmitted from the device 11.

In step S93, by using the acquired position information, the position information calculation unit 141 sets a position information consecutive section, and calculates a distance of the position information consecutive section.

In step S94, the step length calculation unit 142 acquires the number of steps information in the position information consecutive section calculated by the position information calculation unit 141 from the information transmitted from the device 11, and calculates the step length on the basis of the number of steps information in the position information consecutive section.

In step S95, the walking distance and speed calculation unit 143 calculates the walking distance and the walking speed in the position information consecutive section on the basis of the step length calculated by the step length calculation unit 142.

In step S96, the step length calculation unit 142 calculates the amount of change in the atmospheric pressure from the sensor data.

In step S97, the step length calculation unit 142 calculates the height of stairs on the basis of the distance of the position information consecutive section calculated in step S93, the walking distance of the position information consecutive section calculated in step S95, and the amount of change in the atmospheric pressure calculated in step S94.

### <Calculation of Height of Stairs>

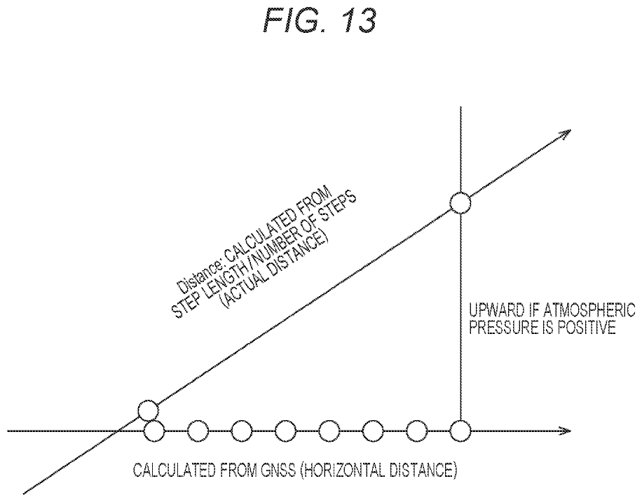

FIG. 13 is a diagram illustrating a calculation method of the height of stairs.

There are a method of calculating the height of stairs on the basis of the sensor data of the atmospheric pressure sensor described above and a method of obtaining the height of stairs using a trigonometric function as follows.

FIG. 13 illustrates a triangle including the horizontal distance of the position information consecutive section acquired from the GNSS, the walking distance of the position information consecutive section, and the height of stairs.

The horizontal distance of the position information consecutive section is obtained in step S93 in FIG. 12. The direction of the height of stairs is obtained from the atmospheric pressure obtained in step S94. The walking distance of the position information consecutive section is obtained in step S95.

Therefore, the height of stairs can be obtained by using a trigonometric function of the triangle illustrated in FIG. 13.

As described above, by calculating the height information, the walking information can be calculated more accurately. Furthermore, there are two types of height calculation methods. By calculating the height by these two types of methods, the accuracy of calculating the height information can be further improved.

### <Functional Configuration of Each Device>

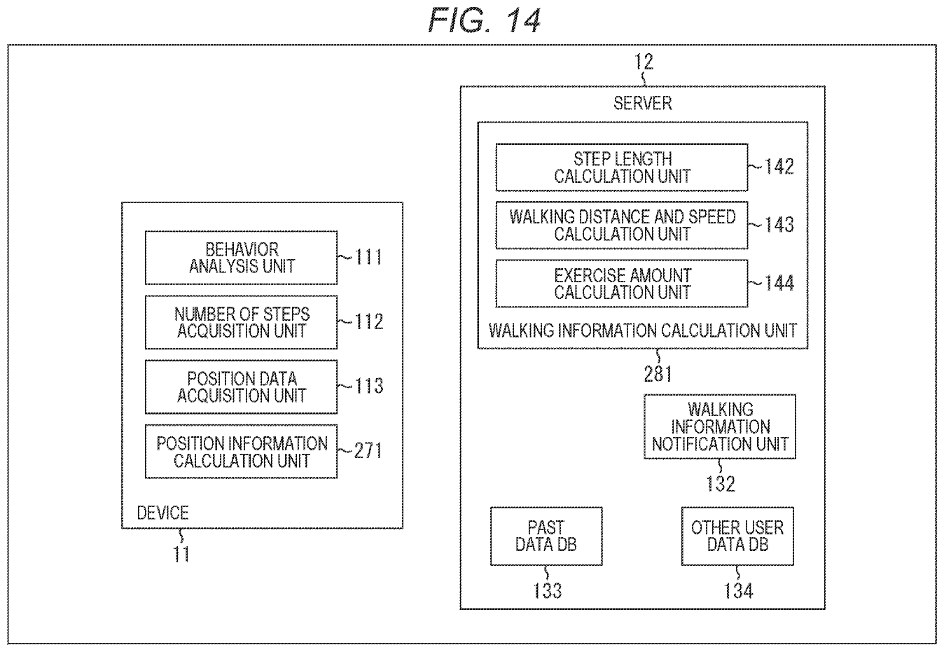

FIG. 14 is a block diagram illustrating another functional configuration example of the device 11 and the server 12.

A device 11 in FIG. 14 is different from the device 11 in FIG. 3 in that a position information calculation unit 271 is added.

A server 12 in FIG. 14 is different from the server 12 in FIG. 3 in that the walking information calculation unit 131 is replaced with a walking information calculation unit 281. The walking information calculation unit 281 is different from the walking information calculation unit 131 in FIG. 3 in that the position information calculation unit 141 is removed.

That is, the position information calculation unit 271 is basically configured similarly to the position information calculation unit 141. That is, the position information calculation unit 271 acquires the position information at the same time as when the behavior pattern information supplied from the behavior analysis unit 111 is Walk or Run. By using the acquired position information, the position information calculation unit 271 sets a position information consecutive section, and calculates a distance of the position information consecutive section.

The position information calculation unit 271 transmits information indicating the set position information consecutive section to the server 12 together with the sensor data.

Although FIG. 14 illustrates an example in which a part of the walking information calculation unit 131 is included in the device 11 and the rest is included in the server 12, all the functional configurations of the server 12 in FIG. 3 may be included in the device 11.

As described above, according to the walking information providing system 1 of the present technology, the walking information indicating the walking state of the user can be more accurately acquired. With this arrangement, for example, the weakening of the walking state of the user can be immediately obtained every day, and thus, the frailty of the user can be prevented.

Note that the checking of the walking state of the user can be used for early detection of the Parkinson's symptom and verification of behavior improvement before and after surgery for cardiopulmonary diseases, in addition to the prevention of frailty. Therefore, the present technology can also be applied to early detection of Parkinson's symptoms and verification of behavior improvement before and after surgery for cardiopulmonary diseases.

### 2. Second Embodiment (Health Information Providing System)

### (Configuration Example of Health Information Providing System)

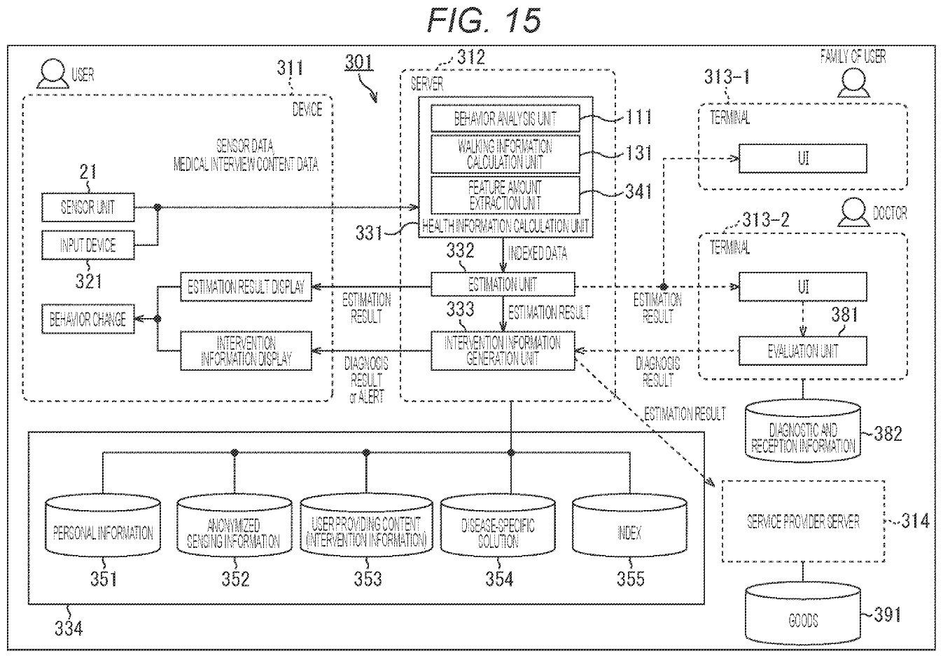

FIG. 15 is a diagram illustrating a configuration example of an embodiment of a health information providing system to which the present technology is applied.

In the health information providing system 301 in FIG. 15, not only the walking information calculated in the first embodiment but also health information indicating a health condition including muscle strength information, motion range information, and the like is acquired, and health information is provided.

That is, the health information providing system 301 in FIG. 15 is a system that calculates the health information including the walking information by using sensor data that is information obtained by sensing the user, and notifies, for example, the user, a family member of the user, a doctor, a service provider, and the like of an estimation result of estimating an amount of change in the calculated health information, the health information itself, and the like. The health information includes, for example, the above-described walking information, muscle strength information, motion range information, and the like that can be calculated from the sensor data. Furthermore, the calculation of the health information is not limited to the calculation using the sensor data, and the calculation thereof may use health information, medical information, or personal information acquired from another database or a terminal device.

The health information providing system 301 includes a device 311, a server 312, terminals 313-1 and 313-2, and a service provider server 314. The device 311, the server 312, the terminals 313-1 and 313-2, and the service provider server 314 are connected via the not-illustrated Internet. The terminals 313-1 and 313-2 are collectively referred to as terminals 313 in a case where the terminals do not need to be distinguished from each other.

Note that, similarly to FIG. 3, the functional configuration illustrated in FIG. 15 is realized by executing a predetermined program by, for example, the CPU 51 illustrated in FIG. 2 included in each of the devices.

The device 311 is different from the device 11 in FIG. 3 in that an input device 321 is added and the behavior analysis unit 111 is removed. Note that, in the example in FIG. 15, the pedometer 112 and the GNSS module 113 in FIG. 3 are configured as a part of a sensor unit 21, and the sensor data transmitted to the server 312 includes the number of steps information and the position information.

The input device 321 includes a touch panel or the like, and is used to input a medical interview content or the like according to the operation of the user.

That is, in the device 311, the sensor data input from the sensor unit 21, the medical interview content data input from the input device 321, and the like are transmitted to the server 312 as they are.

The device 311 displays an estimation result or intervention information transmitted from server 312. The intervention information is content for presenting, to the user, disease-specific alerts, advice, rehabilitation programs, diagnosis results, sales information (including service information) regarding health foods, prescriptions, medicines, and the like.

By viewing the estimation result and the intervention information displayed on the device 311, the user can know a change in one's physical condition, and receive advice, diagnosis, and the like from a doctor, a pharmacist, a health instructor, and the like. Knowing the physical condition and the diagnosis result of the user leads to a behavior change of the user. The behavior change of the user may be input to the device 311 and analyzed by the server 312.

The server 312 includes a health information calculation unit 331, an estimation unit 332, an intervention information generation unit 333, and a database (DB) 334.

The health information calculation unit 331 includes a behavior analysis unit 111, a walking information calculation unit 131, and a feature amount extraction unit 341. That is, the behavior analysis unit 111 included in the device 11 in FIG. 3 is included in the server 312.

That is, the behavior analysis unit 111 performs the behavior analysis and the walking analysis in a case where acceleration has been detected, on the basis of the sensor data and the medical interview content data received from the device 311.

The walking information calculation unit 131 calculates the walking information by using the sensor data and the medical interview content data transmitted from the device 311 and on the basis of the behavior pattern information obtained as a result of analyzing the behavior by the behavior analysis unit 111.

The feature amount extraction unit 341 extracts a feature amount based on any index among indices of a disease, a function, and behavior stored in the index DB 355 from the health information including the walking information calculated by the walking information calculation unit 131. For example, in a case where the feature amount is based on a frailty index, walking information such as a step length is extracted as the feature amount.

The estimation unit 332 estimates a disease, a function, and behavior based on the feature amount extracted by the feature amount extraction unit 341 and the estimation algorithm stored in the index DB 355. The estimation algorithm may be an algorithm for evaluating a disease, a function, or behavior on the basis of a specific threshold value for the feature amount, or may be an algorithm for estimating a disease, a function, or behavior on the basis of a learning model. Here, the threshold value used for estimation may be calculated on the basis of past data of the user or data of other users, or may be determined in advance by a doctor or a service provider.

In a case where the amount of change in the extracted feature amount from the daily feature amount is estimated to be large, the estimation unit 332 transmits information regarding the estimation result and the health information of the user to the device 311, the terminal 313, and the service provider server 314, and outputs the estimation result to the intervention information generation unit 333 to generate the intervention information.

The intervention information generation unit 333 generates intervention information for intervening with the user, on the basis of the estimation result supplied from the estimation unit 332, the diagnosis result transmitted from the terminal 313-2 of the doctor, the sales information transmitted from the service provider server 314, and the like.

The intervention information generation unit 333 transmits the generated intervention information to the device 311.

The DB 334 includes a personal information DB 351, an anonymized sensing information DB 352, an intervention information DB 353, a disease-specific solution DB 354, and an index DB 355.

The personal information DB 351 stores information regarding a user individual such as user's age, user's height, weight, diet, sleep, walking information, muscle strength information, and motion range information. FIG. 15 illustrates the configuration in which the personal information is stored on the server 312 as the database, but the present invention is not limited thereto, and the personal information may be stored, for example, in the device 311 of the user.

The anonymized sensing information DB 352 stores anonymized past sensing data, walking information, and the like of various users used by the health information calculation unit 331 in association with anonymized information such as age, gender, and disease.

The intervention information DB 353 stores the intervention information generated by the intervention information generation unit 343 in association with the health information.

The disease-specific solution DB 354 stores disease-specific solutions such as frailty, Parkinson disease, cardiopulmonary disease, cerebral infarction rehabilitation, and side effect prevention used by the intervention information generation unit 343. In the disease-specific solution DB 354, for example, advice, educational content, a rehabilitation and exercise program, and the like are stored for each disease. Here, each content may be stored in association with the estimation result or the diagnosis result. With this arrangement, for example, an appropriate advice or content can be provided on the basis of the difference from the threshold value in the specific index acquired as the estimation result.

The index DB 355 stores indices of a disease, a function, and behavior used by the estimation unit 332.

Similarly to the terminal 13 in FIG. 3, the terminal 313 includes a mobile terminal, a smartphone, a tablet terminal, a personal computer, or the like.

The terminal 313-1 is carried by a family member of the user. The terminal 313-1 receives and displays the health information of the user, the estimation result, and the like transmitted from the server 312. With this arrangement, even if the family member of the user lives far away from the user, the family member can know the health state of the user and the behavior change of the user.

The terminal 313-2 is carried by a doctor, a pharmacist, or a health consultant. The terminal 313-2 includes an evaluation unit 381 and a diagnostic and reception (medical treatment fee specification) information DB 382. The terminal 313-2 receives and displays the health information of the user, the estimation result, and the like transmitted from the server 312.

The evaluation unit 381 diagnoses the current health condition of the user on the basis of the information input by the doctor or the like, the health information of the user or the estimation result received by the server 312, and the information stored in the diagnostic and reception information DB 382, and transmits the diagnosis result or advice corresponding to the diagnosis result to the server 312. Here, the diagnosis result and the advice corresponding to the diagnosis result may be directly transmitted to the device 311 owned by the user without passing through the server 312.

In the diagnostic and reception information DB 382, diagnostic information, drug information, communication information, genome information, and reception information diagnosed by the user in the past are stored.

As described above, in the terminal 313-2, because the health information of the user, the estimation result, and the like are received, the doctor can know the health state of the user and the behavior change of the user even if the doctor lives far away from the user. With this arrangement, the doctor can remotely diagnose the current health condition of the user and provide the diagnosis result.

The service provider server 314 includes a computer or the like, and includes a goods sales DB 391. The goods sales DB 391 stores goods sales information such as health foods suitable for each piece of health information and the estimation result.

The service provider server 314 receives the health information of the user, the estimation result, and the like transmitted from the server 312, searches the goods sales DB 391 for goods sales information according to the received health information of the user, the estimation result, and the like, and transmits the searched goods sales information to the server 312.

As described above, because the health information of the user, the estimation result, and the like are received in the service provider server 314, the service provider can provide the user with the goods sales information such as the recommended health food on the basis of the health state of the user and the behavior change of the user.

Note that FIG. 15 illustrates a configuration in which the health information is calculated and the estimation result and the intervention information are provided in the server 312. However, the health information providing system 301 may separately include a server that calculates the health information and a server that performs estimation and provides the estimation result and the intervention information. Furthermore, the service provider server 314 has been described as a server different from the server 312 in FIG. 15, but may be managed by the same server as the server 312.

Furthermore, although the example in which the server 312 includes the DB 334 has been described, in the health information providing system 301, a database server including the DB 334 may be separately included. Moreover, each DB in the DB 334 may be managed by different servers.

### <Processing of Health Information Providing System>

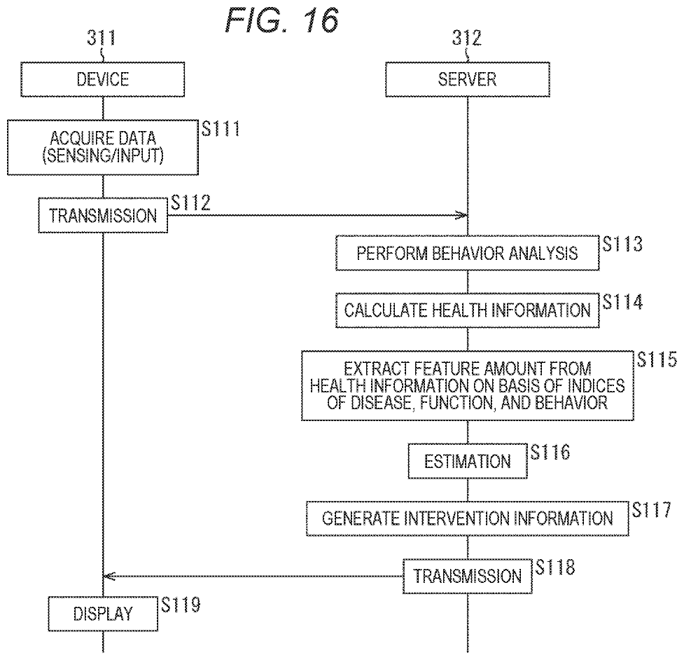

FIG. 16 is a flowchart for explaining processing by the device 311 and the server 312.

First, sensing by the sensor unit 21 and input by the input device 321 are performed. In step S111, the device 311 acquires the sensor data input from the sensor unit 21, the medical interview content data input from the input device 321, and the like and transmits these pieces of data to the server 312 in step S112. Here, the information transmitted to the server 312 is not limited to the sensing and the input by the input device 321 only, and may include information acquired from another database.

In step S113, the behavior analysis unit 111 of the server 312 detects the acceleration on the basis of the sensor data supplied from the sensor unit 21 and performs the behavior analysis and the walking analysis.

In step S114, the walking information calculation unit 131 calculates the walking information among the health information by using the sensor data and the medical interview content data transmitted from the device 311 and on the basis of the behavior analysis information analyzed by the behavior analysis unit 111. Note that other pieces of health information are also calculated using the sensor data and the medical interview content data.

In step S115, the feature amount extraction unit 341 extracts a feature amount on the basis of indices of a disease, a function, and behavior stored in the index DB 355 from the health information including the walking information calculated by the walking information calculation unit 131.

In step S116, the estimation unit 332 compares the feature amount extracted by the feature amount extraction unit 341 with, for example, a daily feature amount to estimate whether or not the amount of change in the feature amount is large.

In step S117, in a case where the amount of change in the feature amount is estimated to be large, the estimation unit 332 outputs the estimation result to the intervention information generation unit 333 to cause the intervention information to be generated. The intervention information generation unit 333 generates intervention information for intervening with the user, on the basis of the estimation result supplied from the estimation unit 332, the diagnosis result transmitted from the terminal 313-2 of the doctor, the sales information transmitted from the service provider server 314, and the like.

In step S118, the intervention information generation unit 333 transmits the generated intervention information to the device 311.

In step S119, the device 311 displays the intervention information including the estimation result, the diagnosis result, or the alert transmitted from the server 312.

The user can know the change in one's physical condition by viewing the estimation result and the intervention information displayed on the device 311. By knowing one's own physical condition, a change appears in the behavior of the user.

### <Configuration Example of Health Information Providing System>

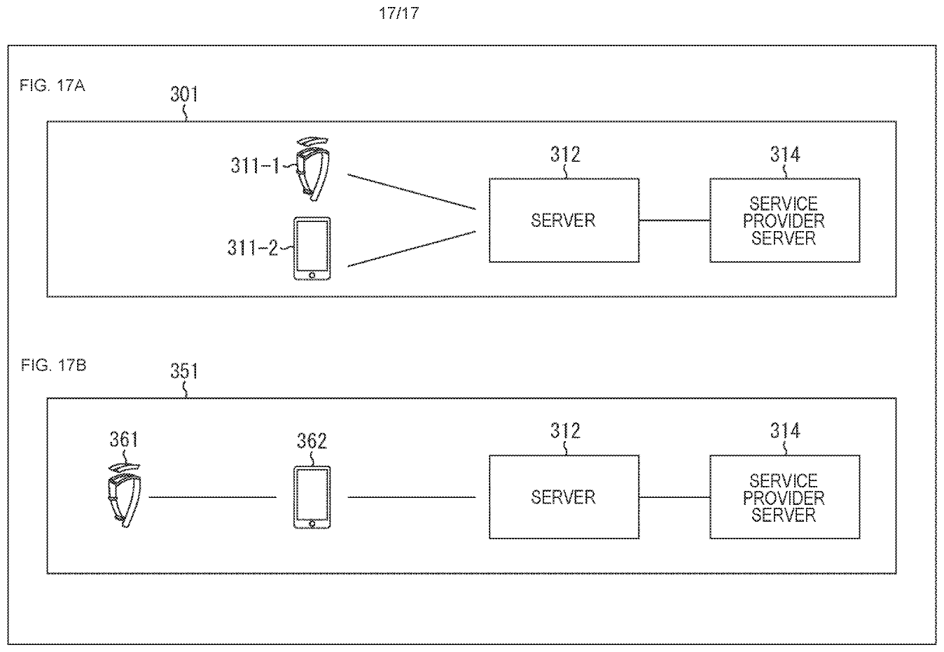

FIG. 17 is a diagram illustrating a configuration example of a health information providing system.

The health information providing system may have a configuration illustrated in A of FIG. 17 or a configuration illustrated in B of FIG. 17.

Similarly to FIG. 15, a health information providing system 301 illustrated in A of FIG. 17 is configured such that a device 311-1 which is a sensor terminal including a sensor and a device 311-2 which is a portable terminal such as a smartphone are directly connected to a server 312, and the server 312 and a service provider server 314 are directly connected to each other.

A health information providing system 361 illustrated in B of FIG. 17 is configured such that a device 361-1 which is a sensor terminal including a sensor temporarily accumulates and compiles sensor data in a device 361-2 which is a portable terminal such as a smartphone, and then transmits the accumulated sensor data to the server 312.

Note that, in A of FIG. 17 and B of FIG. 17, examples in which the service provider server 314 is connected to the server 312 is illustrated, but the service provider server 314 is not necessarily a server and may be a terminal of a service provider.

### 3. Others

### Effect

As described above, in the present technology, the walking information regarding walking of the user is calculated by using the position information of the consecutive section set on the basis of the behavior analysis information of the user.

Therefore, according to the present technology, the walking information of the user can be acquired more accurately.

With this arrangement, in a case where the user is an elderly person, because the sign of frailty of the user can be noticed at an early stage, it is possible to take measures against frailty and prevent frailty.

### (Configuration Example of Computer)

The above-described series of processing can be executed by hardware or can be executed by software. In a case where the series of the processing is executed by software, a program included in the software is installed from a program recording medium to a computer incorporated in dedicated hardware or a general-purpose personal computer.

Note that the program executed by the computer may be a program that executes processing in time series in the order described in the present description, or a program that executes processing in parallel or at a necessary timing such as when a call is made.

Note that, in the present description, a system means an assembly of a plurality of constituents (devices, modules (components), and the like), and it does not matter whether or not all the constituents are located in the same housing. Therefore, a plurality of devices housed in separate housings and connected via a network and one device in which a plurality of modules is housed in one housing are both systems.

Furthermore, the effects described in the present description are merely examples and not restrictive, and there may also be other effects.

The embodiment of the present technology is not limited to the above-described embodiments, and various modifications may be made without departing from the gist of the present technology.

For example, the present technology may be configured as cloud computing in which one function is shared by a plurality of devices via a network and processing is executed in cooperation.

Furthermore, each of the steps in the flowcharts described above can be executed by one device or executed by a plurality of devices in a shared manner.

Moreover, in a case where a plurality of types of processing is included in one step, the plurality of types of processing included in one step can be executed by one device or by a plurality of devices in a shared manner.

### Combination Examples of Configurations

The present technology may also have the following configurations.

(1)

An information processing apparatus including
a walking information calculation unit that calculates walking information indicating a walking state of a user by using position information of a consecutive section set on the basis of behavior analysis information obtained as a result of analyzing behavior of the user.
   (2)

The information processing apparatus according to (1), in which
the walking information calculation unit calculates at least one of a step length, the number of steps, a walking distance, a walking speed, or an exercise amount of the user.
   (3)

The information processing apparatus according to (1) or (2), in which
the walking information calculation unit calculates the walking information on the basis of position information of the consecutive section on which excluding processing has been performed.
   (4)

The information processing apparatus according to any one of (1) to (3), in which
the walking information calculation unit calculates the walking information of a section in which position information of the consecutive section cannot be acquired by interpolation processing based on the walking information that has been calculated.
   (5)

The information processing apparatus according to any one of (1) to (4), in which
the walking information calculation unit calculates the walking information on the basis of external environment information of the consecutive section.
   (6)

The information processing apparatus according to any one of (1) to (5), in which
the walking information calculation unit calculates the walking information on the basis of height information estimated from atmospheric pressure sensor data.
   (7)

The information processing apparatus according to any one of (1) to (6), in which
the walking information calculation unit calculates the walking information on the basis of height information calculated from a distance of the position information of the consecutive section and the number of steps and the step length of the user.
   (8)

The information processing apparatus according to (1), further including
a storage unit that stores the walking information that has been calculated.
   (9)

The information processing apparatus according to (8), in which
the storage unit stores the walking information in association with at least one of the position information of the consecutive section or the behavior analysis information.
   (10)

The information processing apparatus according to (9), in which
the walking information calculation unit calculates the walking information of a section in which the position information of the consecutive section cannot be acquired, by interpolation processing based on the walking information of oneself or other users stored in the storage unit.
   (11)

The information processing apparatus according to any one of (1) to (10), further including
a position information setting unit that sets, in a case where a specific behavior has been analyzed on the basis of the behavior analysis information, position information of a consecutive section in which the specific behavior has been analyzed.
   (12)

The information processing apparatus according to (11), in which
the position information setting unit performs excluding processing on the position information of the consecutive section that has been set.
   (13)

The information processing apparatus according to any one of (1) to (12), further including
a transmission unit that transmits the walking information that has been calculated to an information processing terminal.
   (14)

The information processing apparatus according to (13), in which
the transmission unit transmits the walking information to the information processing terminal in a case where an amount of change between the walking information that has been calculated and the walking information in the past is larger than a predetermined threshold value.
   (15)

The information processing apparatus according to (14), in which
the predetermined threshold value is a value different for each user of the information processing terminal.
   (16)

An information processing method including
calculating, by an information processing apparatus, walking information indicating a walking state of a user by using position information of a consecutive section set on the basis of behavior analysis information of the user.
   (17)

A program causing a computer to function as
a walking information calculation unit that calculates walking information indicating a walking state of a user by using position information of a consecutive section identified on the basis of behavior analysis information of the user.
   (18)

An information processing apparatus including:
a behavior analysis unit that analyzes behavior of a user; and
a transmission unit that, in a case where specific behavior has been analyzed, sets position information of a consecutive section in which the specific behavior has been analyzed, and transmits the position information to another information processing apparatus.
   (19)

An information processing system including:
a first information processing apparatus including
a behavior analysis unit that analyzes behavior of a user, and
a transmission unit that sets, in a case where specific behavior has been analyzed, position information of a consecutive section in which the specific behavior has been analyzed and transmits the position information to a second information processing apparatus; and
the second information processing apparatus including
a walking information calculation unit that calculates walking information indicating a walking state of the user by using the position information of the consecutive section set by the first information processing apparatus.

### REFERENCE SIGNS LIST

1 Walking information providing system
11 Device
12 Server
13-1, 13-2, 13 Terminal
14 Internet
21 Sensor unit
51 CPU
111 Behavior analysis unit
112 Pedometer
113 GNSS module
131 Walking information calculation unit
132 Walking information notification unit
133 Past data DB
134 Other user data DB
141 Position information calculation unit
142 Step length calculation unit
143 Walking distance and speed calculation unit
144 Exercise amount calculation unit
201 Walking information calculation unit
211 Area section extraction unit
212 Inclination distance calculation unit
251 Walking information calculation unit
262 Height estimation unit
271 Position information calculation unit
281 Walking information calculation unit
301 Health information providing system
311, 311-1, 311-2 Device
312 Server
313-1, 313-2, 313 Terminal
314 Service provider server
321 Input device
331 Health information calculation unit
332 Estimation unit
333 Intervention information generation unit
334 Database
341 Feature amount extraction unit
351 Personal information DB
352 Anonymized sensing information DB
353 Intervention information DB
354 Disease-specific solution DB
355 Index DB
381 Evaluation unit
382 Diagnostic and reception information DB
391 Goods sales DB
351 Health information providing system
361 Device

1. An information processing apparatus comprising
a walking information calculation unit that calculates walking information indicating a walking state of a user by using position information of a consecutive section set on a basis of behavior analysis information obtained as a result of analyzing behavior of the user.

2. The information processing apparatus according to claim 1, wherein
the walking information calculation unit calculates at least one of a step length, a number of steps, a walking distance, a walking speed, or an exercise amount of the user.

3. The information processing apparatus according to claim 1, wherein
the walking information calculation unit calculates the walking information on a basis of position information of the consecutive section on which excluding processing has been performed.

4. The information processing apparatus according to claim 1, wherein
the walking information calculation unit calculates the walking information of a section in which position information of the consecutive section cannot be acquired by interpolation processing based on the walking information that has been calculated.

5. The information processing apparatus according to claim 1, wherein
the walking information calculation unit calculates the walking information on a basis of external environment information of the consecutive section.

6. The information processing apparatus according to claim 1, wherein
the walking information calculation unit calculates the walking information on a basis of height information estimated from atmospheric pressure sensor data.

7. The information processing apparatus according to claim 1, wherein
the walking information calculation unit calculates the walking information on a basis of height information calculated from a distance of the position information of the consecutive section and a number of steps and a step length of the user.

8. The information processing apparatus according to claim 1, further comprising
a storage unit that stores the walking information that has been calculated.

9. The information processing apparatus according to claim 8, wherein
the storage unit stores the walking information in association with at least one of the position information of the consecutive section or the behavior analysis information.

10. The information processing apparatus according to claim 9, wherein
the walking information calculation unit calculates the walking information of a section in which the position information of the consecutive section cannot be acquired, by interpolation processing based on the walking information of oneself or other users stored in the storage unit.

11. The information processing apparatus according to claim 1, further comprising
a position information setting unit that sets, in a case where a specific behavior has been analyzed on a basis of the behavior analysis information, position information of a consecutive section in which the specific behavior has been analyzed.

12. The information processing apparatus according to claim 11, wherein
the position information setting unit performs excluding processing on the position information of the consecutive section that has been set.

13. The information processing apparatus according to claim 1, further comprising
a transmission unit that transmits the walking information that has been calculated to an information processing terminal.

14. The information processing apparatus according to claim 13, wherein
the transmission unit transmits the walking information to the information processing terminal in a case where an amount of change between the walking information that has been calculated and the walking information in a past is larger than a predetermined threshold value.

15. The information processing apparatus according to claim 14, wherein
the predetermined threshold value is a value different for each user of the information processing terminal.

16. An information processing method comprising
calculating, by an information processing apparatus, walking information indicating a walking state of a user by using position information of a consecutive section set on a basis of behavior analysis information of the user.

17. A program causing a computer to function as
a walking information calculation unit that calculates walking information indicating a walking state of a user by using position information of a consecutive section identified on a basis of behavior analysis information of the user.

18. An information processing apparatus comprising:
a behavior analysis unit that analyzes behavior of a user; and
a transmission unit that, in a case where specific behavior has been analyzed, sets position information of a consecutive section in which the specific behavior has been analyzed, and transmits the position information to another information processing apparatus.

19. An information processing system comprising:
a first information processing apparatus including a behavior analysis unit that analyzes behavior of a user, and
a transmission unit that sets, in a case where specific behavior has been analyzed, position information of a consecutive section in which the specific behavior has been analyzed and transmits the position information to a second information processing apparatus; and
the second information processing apparatus including
a walking information calculation unit that calculates walking information indicating a walking state of the user by using the position information of the consecutive section set by the first information processing apparatus.
